(19) United States
(12) Patent Application Publication
(10) Pub. No.: US 2009/0093641 A1
(43) Pub. Date: Apr. 9, 2009

(54) Synthesis of Pentafluorosulfanyl (SF5)-Substituted Heterocycles and Alkynes

(75) Inventors: William R. Dolbier, JR., Gainesville, FL (US); Akira Mitani, Gainesville, FL (US)

Correspondence Address: SALIWANCHIK LLOYD & SALIWANCHIK;A PROFESSIONAL ASSOCIATION, PO Box 142950, GAINESVILLE, FL, 32614, US

(73) Assignee: UNIVERSITY OF FLORIDA RESEARCH FOUNDATION, INC., Gainesville, FL (US)

(21) Appl. No.: 12/282,753

(22) PCT Filed: Mar. 13, 2007
(86) PCT No.: PCT/US2007/063898

§ 371 Date: Sep. 12, 2008

Related U.S. Application Data
(60) Provisional application No. 60/781,817, filed on Mar. 13, 2006.

(51) Int. Cl. C07D 261/04; C07D 307/64; C07D 493/08

(52) U.S. Cl. 548/243; 549/479; 549/463

(57) ABSTRACT

The subject invention pertains to pentafluorosulfanyl (—SF₅) substituted compounds and methods of their synthesis. The subject invention provides convenient methods to incorporate the —SF₅ substituent into compounds such as heterocycles and alkynes.

### CROSS-REFERENCE TO RELATED APPLICATION

This application claims the benefit of U.S. Provisional Application Ser. No. 60/781,817, filed Mar. 13, 2006, the disclosure of which is hereby incorporated by reference in its entirety.

### BACKGROUND OF INVENTION

There is currently great interest in methods for the preparation of selectively fluorinated organic compounds. This interest results from the profound influence that fluorine incorporation can have on the physical properties, chemical properties, and biological activity of molecules. For example, methods for putting the bulky, highly electronegative and generally inert trifluoromethyl group into organic compounds have received much research attention during recent years.

Another fluorinated substituent that could attract interest among synthetic organic chemists is the pentafluorosulfanyl (SF₅) group (Winter et al., Inorganic Fluorine Chemistry-Toward the 21st Century (1994) 555:128-47, Pub: American Chemical Society: Washington (Thrasher, J. S., Strauss, S. H., Eds.); Lentz et al., Chemistry of Hypervalent Compounds (1999) 295-326; Pub: Wiley-VCH: New York (Akiba, K., Ed.); Verma et al., Advances in Inorganic Chemistry (1994) 41:125-69, Pub: Academic Press: San Diego (Sykes, A. G., Ed.); pentafluorosulfanyl groups bear some similarity to trifluoromethyl groups, however, SF₅ is more electronegative (σₚ=+0.68 versus +0.54 for CF₃; Sheppard, W. A., J. Am. Chem. Soc. (1962) 84:3072-6) and more sterically demanding.

However, until the development of the subject invention, many methods required the use of elemental F₂ or oxidative fluorination by AgF₂ (Sheppard, W. A., J. Am. Chem. Soc. (1962) 84:3064-3072; Chambers et al., Chem. Commun. (1999) 883-884; Bowden et al., Tetrahedron (2000) 56:3399-3408; Sipyagin et al., J. Fluorine Chem. (2001) 112:287-295) to incorporate an SF₅ group into aliphatic compounds (that is, the methodologies relied on high pressure autoclave or specialized photochemical procedures) (Case et al., J. Chem. Soc. (1961) 2066-2070; Wessel et al., Chem. Ber. (1983) 116:2399-2407; Winter et al., J. Fluorine Chem. (1994) 66:109-116; Fokin et al., Russ. Chem. Bull. (1996) 45:2804-6). U.S. Pat. No. 6,919,484 provided methods for incorporating an —SF₅ group into alkanes, alkenes, and aromatics by condensing SF₅Cl into a hexane solution that also contains the alkane, alkene, or aromatic of interest. However, the introduction SF₅ into alkynyl and heterocyclic compounds has not been widely practiced by synthetic organic chemists.

SF₅Cl is presently the only commercially available “reagent” that can be used to introduce the SF₅ substituent into aliphatic compounds. As a gaseous pseudo halogen, this reagent cannot be used as an electrophilic source of SF₅. It has, however, been used in free radical chain alkene/alkyne addition processes (Sidebottom et al., Trans. Faraday Soc. (1969) 65:2103-2109). These processes are generally done thermally, in an autoclave, with or without an initiator, or using room temperature gas phase or low temperature solution phase photochemical processes. For example (Case et al., J. Chem. Soc. (1961) 2066-2070):

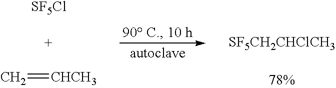

In order for SF₅-derivatives to become incorporated into the day-to-day strategic planning of working synthetic organic chemists, a convenient bench-top procedure for the introduction of SF₅ substituents into organic substrates is needed. The subject invention provides such a method—one that will allow convenient addition of SF₅Cl to a large variety of heterocyclic and alkynyl compounds in excellent yield.

### BRIEF SUMMARY OF THE INVENTION

One aspect of the subject invention relates to novel pentafluorosulfanyl containing compounds. Specifically, the subject invention relates to pentafluorosulfanyl substituted heterocycles, alkynes, and intermediate products of the processes of the subject invention along with analogues of each of the aforementioned compounds. Exemplary compounds of the subject invention include, without limitation, 3-pentafluorosulfanylfuran, 2-methyl-4-pentafluorosulfanylfuran, 3-pentafluoro-sulfanyl-4-butylfuran, 3-phenyl-4-pentafluorosulfanyl-5-butyl-isoxazole, 2,3-diphenyl-4-pentafluorosulfanyl-5-butylisooxazoline, 3,5-diphenyl-4-pentafluorosulfanylisooxazole, 4-pentafluorosulfanyl-2,3,4-tripenyl-4-isooxazoline, and 2-butyl-3-pentafluorosulfanylbicyclo[2.2.1]hepta-2,5-diene.

Another aspect of the subject invention pertains to processes used to synthesize heterocycles and alkynes substituted with a pentafluorosulfanyl group along with any pentafluorosulfanyl substituted intermediate products of the subject processes.

### DETAILED DISCLOSURE OF THE INVENTION

The subject invention provides pentafluorosulfanyl (SF₅) substituted heterocyclic and alkynyl compounds, their analogues, and processes for their preparation.

The simplicity of the new processes provided by the subject invention, combined with the generally excellent yields that are obtained, constitutes a breakthrough in SF₅ synthetic methodology that opens the door to the convenient, bench top preparation of a multitude of SF₅-containing heterocyclic and alkynyl compounds by synthetic organic chemists. Thus, the subject invention has application to broad applicability to any compound containing heterocyclic or alkynyl groups, including functionalized or substituted compounds.

Compounds of the Subject Invention Include, without Limitation:

compound (1):

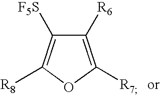

compound (2):

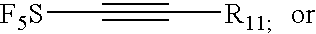

compound (3):

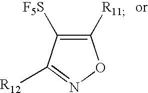

compound (4):

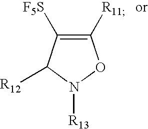

compound (5):

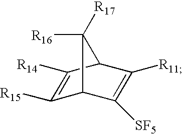

wherein R₆, R₇, R₈, R₁₁, R₁₂, R₁₃, R₁₄, R₁₅, R₁₆, and R₁₇ are, independently, hydrogen, unsubstituted alkyl, substituted alkyl, unsubstituted alkenyl, disubstituted alkenyl, substituted alkenyl, unsubstituted alkynyl, substituted alkyl, unsubstituted aryl, substituted aryl, substituted or unsubstituted substituted or unsubstituted trialkylsilyl, cycloalkanes, cycloalkenes, cyclodialkenes, hydroxy, alkoxy, ether, ketone, carboxyl, aldehyde, amino, thio, phosphate, or halogen.

In a specific embodiment, R₆, R₇, and R₈ of compound (1) are each hydrogen. In yet another specific embodiment, R₆ and R₈ of compound (1) are each hydrogen while R₇ is an unsubstituted alkyl group, preferably a methyl group.

In a specific embodiment for compound (2), R₁₁ is hydrogen, substituted or unsubstituted substituted or unsubstituted trialkylsilyl, an unsubstituted alkyl, preferably butyl, or an unsubstituted aryl, preferably a phenyl group.

R₁₁ and R₁₂ of compound (3) are each hydrogen in one embodiment. In yet another embodiment, R₁₁ and R₁₂ have the same substituent group, preferably hydrogen, substituted or unsubstituted trialkylsilyl, or an unsubstituted or substituted alkyl or aryl, more preferably butyl or phenyl. In another specific embodiment, R₁₁ and R₁₂ are each different substituents. For example, R₁₁ is an unsubstituted alkyl group or substituted or unsubstituted trialkylsilyl, and R₁₂ is an unsubstituted phenyl group, or vice versa.

Regarding the substituents of compound (4) of the subject invention, R₁₁ and R₁₂ have the same substituent group, and R₁₃ has a different substituent group in one embodiment. For example, R₁₁ and R₁₂ may each be substituted or unsubstituted trialkylsilyl, an unsubstituted or substituted alkyl, and R₁₃ may be hydrogen. Preferably, R₁₁ and R₁₂ are each substituted or unsubstituted trialkylsilyl, butyl or phenyl, and R₁₃ is hydrogen. In another specific embodiment, R₁₁, R₁₂, and R₁₃ are each different substituents. For example, R₁₁ is an unsubstituted alkyl group or substituted or unsubstituted trialkylsilyl, R₁₂ is an unsubstituted phenyl group, and R₁₃ is hydrogen, or R₁₁ is phenyl or substituted or unsubstituted trialkylsilyl, R₁₂ is alkyl, and R₁₃ is hydrogen. In yet another specific embodiment, R₁₁ and R₁₃ may be the same substituent, and R₁₂ may be different. R₁₂ and R₁₃ may be the same substituent, and R₁₁ may be a different substituent in another embodiment.

Preferably, R₁₁ of compound (5) is hydrogen, substituted or unsubstituted trialkylsilyl, an unsubstituted alkyl, preferably butyl, or an unsubstituted aryl, preferably a phenyl group, and R₁₄, R₁₅, R₁₆, and R₁₇ are each hydrogen.

As used herein, the term “substituted” is used to refer to a functional group substituent like a halogen, an aliphatic chain, an aryl, a ketone, an ether, a hydroxy, an alkoxy, an amino, an aldehyde, a carboxyl group, a phosphate, or a thio.

In a specific embodiment, the preferred compounds include 3-pentafluorosulfanylfuran, 2-methyl-4-pentafluorosulfanylfuran, 3-pentafluoro-sulfanyl-4-butylfuran, 3-phenyl-4-pentafluorosulfanyl-5-butyl-isoxazole, 2,3-diphenyl-4-pentafluorosulfanyl-5-butylisooxazoline, 3,5-diphenyl-4-pentafluorosulfanylisooxazole, 4-pentafluorosulfanyl-2,3,4-tripenyl-4-isooxazoline, and 2-butyl-3-pentafluorosulfanylbicyclo[2.2.1]hepta-2,5-diene.

Another aspect of the subject invention is directed to the synthesis of the compounds of the subject invention. In one embodiment, an SF₅ substituted alkene is converted to an SF₅ substituted alkyne, as shown below in Scheme I,

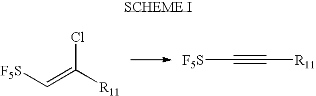

wherein Scheme I takes place in the presence of DMSO and lithium hydroxide monohydrate. The SF₅ substituted alkene is prepared according to the methods of U.S. Pat. No. 6,919,484, which is herein incorporated by reference in its entirety. R₁₁ is a substituent selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, alkoxy, ether, ketone, carboxyl, aldehyde, amino, thio, phosphate, or halogen. In one specific embodiment, R₁₁ is a butyl group or substituted or unsubstituted trialkylsilyl. In yet another specific embodiment, R₁₁ is a phenyl group. In another embodiment, R₁₁ is hydrogen.

The elimination reaction of Scheme I includes contacting the substituted or substituted alkenyl compound with lithium hydroxide monohydrate. The contacting between the two reagents can be enhanced by stirring the mixture for a sufficient amount of time for the reaction to initiate and in some instances, proceed to completion or equilibrium. In a preferred embodiment, the mixture is stirred for about 2 hours. In one embodiment, the contacting step advantageously takes place at room temperature (typically about 20° C. to about 23° C.).

The processes of the subject invention also pertain to reacting the SF₅ substituted alkynyl prepared in Scheme I with the appropriate reactants and/or at least one radical initiator to prepare SF₅ substituted azole, azoline, bridge ring, or furan compounds. An azole compound (3) of the subject invention is prepared by contacting a solution of an SF₅ substituted alkyne in a solvent, preferably tetrahydrofuran, with a R₁₂ substituted hydroxyiminoyl chloride and at least one radical initiator as shown in Scheme II,

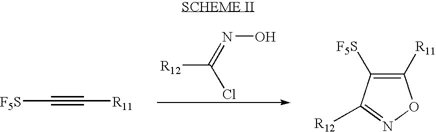

wherein R₁₁ and R₁₂ are substituents independently selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, alkoxy, ether, ketone, carboxyl, aldehyde, amino, thio, phosphate, or halogen. R₁₁ and R₁₂ are preferably and independently hydrogen, substituted or unsubstituted trialkylsilyl, unsubstituted alkyl, preferably butyl, or unsubstituted aryl, preferably phenyl.

The radical initiator is selected from a group consisting of dialkylboranes, trialkylboranes, Et₃N, Et₃N-nHF, and 9-boracicyclo[3.3.1.]nonane, and mixtures of any of the foregoing. Preferably, the initiator is Et₃N, which is added to the alkynyl/R₁₂ substituted hydroxyiminoyl chloride solution in a dropwise fashion. In one embodiment, when the initiator is Et₃N, then it is combined with a solvent to form a solution before addition to the reaction mixture. The preferred solvent for an Et₃N solution is tetrahydrofuran (THF).

The contacting step is optionally enhanced by stirring or other forms of mixing for a sufficient amount of time for the reaction to initiate, or in some instances, to proceed to completion or to equilibrium. In one embodiment, additional amounts of both the R₁₂ substituted hydroxyiminoyl chloride and the Et₃N initiator are added periodically. For example, additional amounts are added at the sixteen and the twenty hour marks. Advantageously, the processes for producing SF₅ substituted azole compounds takes place at room temperature (about 20° C. to about 23° C.).

The processes of the subject invention also pertain to the synthesis of an SF₅ substituted furan compound from an SF₅ substituted alkyne as shown in Scheme III,

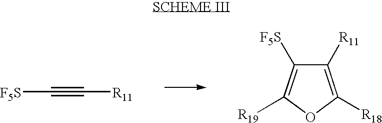

which takes place in the presence of an azole compound, preferably 4-phenyloxazole, and heat. R₁₁, R₁₈, and R₁₉ are each substituents independently selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, alkoxy, ether, ketone, carboxyl, aldehyde, amino, thio, phosphate, or halogen. Preferably, R₁₈ and R₁₉ are each hydrogen. The preferred R₁₁ substituent is hydrogen, substituted or unsubstituted trialkylsilyl, unsubstituted alkyl, preferably butyl, or unsubstituted aryl, preferably phenyl.

In one embodiment, the reaction mixture of the SF₅ substituted alkyne and the azole compound are heated to a temperature between about 170° C. to about 200° C., preferably about 180° C. and about 190° C. The heated temperature is maintained for a sufficient amount of time for the reaction to proceed to completion or equilibrium, preferably about 20 hours.

Another aspect of the processes of the subject invention pertains to the synthesis of SF₅ substituted compounds like compound (4) of the subject invention. As illustrated in Scheme IV, the subject method advantageously utilizes the SF₅ substituted alkyne (compound (2)) produced by Scheme I. Preferably, the SF₅ substituted alkyne (compound (2)) is in solution with a solvent preferably, tetrahydrofuran. This embodiment of the processes of the subject invention comprises contacting a solution of compound (2) in a solvent, preferably tetrahydrofuran, with an R₁₂, R₁₃-disubstituted aniline N-oxide and stirring at room temperature (about 20° C. to about 23° C.) for a sufficient period of time to initiate a reaction, or in some instances, until the reaction reaches completion or equilibrium. Preferably, the reaction mixture is stirred for about 16 hours. Scheme IV is illustrated below:

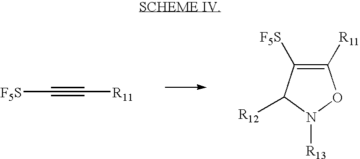

R₁₁, R₁₂, and R₁₃ are substituents independently selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, alkoxy, ether, ketone, carboxyl, aldehyde, amino, thio, phosphate, or halogen. R₁₁ and R₁₂ have the same substituent group, and R₁₃ has a different substituent group. For example, R₁₁ and R₁₂ may each be substituted or unsubstituted trialkylsilyl or an unsubstituted or substituted alkyl, and R₁₃ may be a hydrogen. Preferably, R₁₁ and R₁₂ are each butyl or phenyl, and R₁₃ is hydrogen. In another specific embodiment, R₁₁, R₁₂, and R₁₃ are each different substituents. For example, R₁₁ is an unsubstituted alkyl group or substituted or unsubstituted trialkylsilyl, R₁₂ is an unsubstituted phenyl group, and R₁₃ is hydrogen, or R₁₁ is phenyl or substituted or unsubstituted trialkylsilyl, R₁₂ is alkyl, and R₁₃ is hydrogen. In yet another specific embodiment, R₁₁ and R₁₃ may be the same substituent, and R₁₂ may be different. R₁₂ and R₁₃ may be the same substituent, and R₁₁ may be a different substituent in another embodiment.

Compound (5) of the subject invention is synthesized by Scheme V of the processes of the subject invention. Briefly, an SF₅ substituted alkyne is reacted under heat with a substituted or unsubstituted cyclopentiadiene. In one embodiment, the reaction mixture is heated to a temperature within the range of 100° C. to about 130° C., preferably about 120° C., and maintained at that temperature for a sufficient period of time for the reaction to initiate and in some instances, proceed to completion or equilibrium.

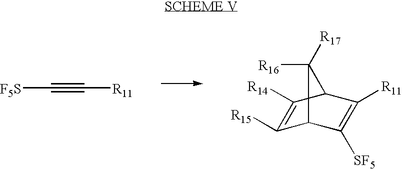

R₁₁, R₁₄, R₁₅, R₁₆, and R₁₇, are each independently selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, alkoxy, ether, ketone, carboxyl, aldehyde, amino, thio, phosphate, or halogen. Preferably, R₁₁ of compound (5) is hydrogen, substituted or unsubstituted trialkylsilyl, an unsubstituted alkyl, preferably butyl, or an unsubstituted aryl, preferably a phenyl group, and R₁₄, R₁₅, R₁₆, and R₁₇ are each hydrogen.

The processes of the subject invention also pertain to synthesizing SF₅ substituted furans using bridged ring compounds as the reactants. In another embodiment, bridged ring compounds, wherein the two rings have two atoms in common, are mixed with SF₅Cl gas at a depressed temperature, for example, about −40° C. The method also comprises contacting the bridged ring compounds and SF₅Cl with one or more radical initiators. The radical initiators are selected from a group consisting of dialkylboranes, trialkylboranes, Et₃N, Et₃N-nHF, and 9-boracicyclo[3.3.1.]nonane, and mixtures of any of the foregoing. Preferably, the initiator is Et₃B. In a preferred contacting step, the radical initiator is added to the reaction mixture in a dropwise manner. Optionally, additional amounts of SF₅Cl and/or Et₃B are periodically added to the reaction mixture.

A suitable bridge ring compound useful as a reactant in this embodiment of the subject processes is:

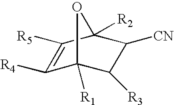

wherein R₁-R₅ are each substituents independently selected from hydrogen, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen. In a one embodiment, R₁-R₅ are each hydrogen. An enantiomeric mixture of the first intermediate SF₅ substituted compound shown below is prepared:

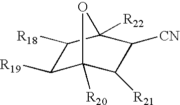

wherein R₁₈ is Cl or SF₅, R₁₉ is Cl or SF₅, and R₁₈ and R₁₉ are not the same substituent; and wherein R₂₀, R₂₁, and R₂₂ are each independently hydrogen, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen.

The resulting SF₅ substituted intermediate product undergoes further reaction with lithium hydroxide monohydrate in the presence of DMSO, thereby eliminating the halogen substituent on either R₁₈ and R₁₉. This elimination takes place advantageously at room temperature (about 20° C. to about 23° C.) and involves the adequate mixing of the intermediate products and the additional reagents. Preferably, the mixing mechanism is stirring. The resulting second intermediate SF₅ substituted intermediate product is:

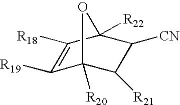

wherein R₁₈ is hydrogen or SF₅, R₁₉ is hydrogen or SF₅, and R₁₈ and R₁₉ are not the same substituent. R₂₀, R₂₁, and R₂₂ each remain independently hydrogen, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, alkoxy, ether, ketone, carboxyl, aldehyde, amino, thio, phosphate, or halogen.

The second intermediate SF₅ substituted product is further processed with a heat treatment, thereby converting the bridge ring compound to a furan compound (1) of the subject invention, followed by a cooldown to room temperature (about 20° C. to about 23° C.).

The various enantiomeric forms of the compounds and intermediate compounds of the subject invention may be isolated according to methods well known to the skilled artisan.

Optionally, the SF₅ substituted compounds and intermediate products prepared according to the processes of the subject invention can undergo neutralization, extraction, washing, purification, and/or distillation using techniques known in the art. Preferably, any neutralization is performed with the addition of a sufficient amount of sodium bicarbonate (HNaCO₃) or acid, preferably hydrochloric acid (HCl). Any drying is preferably performed over a suitable desiccant, for example, MgSO₄. The compounds may be purified by passing through a separation column, for example, a silica gel column, to remove contaminants like unreacted reagents and intermediate products. Purity and/or analysis of the compounds of the subject invention may be determined using techniques known in the art including without limitation NMR analysis. For the intermediate products, any neutralization, extraction, washing, purification, and/or distillation takes place before the next reaction sequence. In the above-described compounds and intermediate products of the subject invention, bond line notation has been used. Thus, the skilled artisan would understand that although not always depicted, hydrogen atoms are present in an amount to satisfy the requirement that each carbon atom has four bonds.

The terms “comprising”, “consisting of”, and “consisting essentially of” are defined according to their standard meaning and may be substituted for one another throughout the instant application in order to attach the specific meaning associated with each term.

As used in this specification and the appended claims, the singular forms “a”, “an”, and “the” include plural reference unless the context clearly dictates otherwise. Thus, for example, a reference to “an alkyne” includes more than one such alkyne, a reference to “the method” includes more than one such method, and the like.

The subject invention also provides the following non-limiting embodiments:

1. A compound selected from:

compound (1):

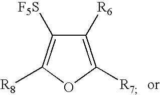

compound (2):

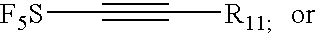

compound (3):

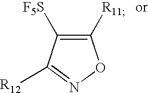

compound (4):

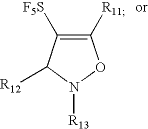

compound (5):

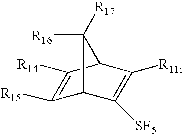

wherein R₆, R₇, R₈, R₁₁, R₁₂, R₁₃, R₁₄, R₁₅, R₁₆, and R₁₇ are, independently, hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen;

with the proviso that compound (2) excludes:

1) compounds of the formula:

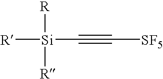

or liquid crystals thereof;

wherein:

i) substituents R, R′, and R″ are selected from the group consisting of an alkyl, a substituted alkyl, an aryl, a substituted aryl, or combinations thereof and at least one of the substituents is hindered;

ii) R, R′, or R″ comprises an alkyl and at least one of the substituents is hindered;

iii) at least one of R, R′, or R″ comprises t-butyl and at least one of the substituents is hindered;

iv) R and R′ comprises CH₃ and R″ comprises t-butyl and at least one of the substituents is hindered;

v) at least one of R, R′, or R″ comprises isopropyl and at least one of the substituents is hindered; and/or

vi) the molecular weight of the compound ranges from about 225 to about 800 or about 225 to 400; or

2) compounds or liquid crystals thereof comprising:

i) a sulfurpentafluoride group and a substituted silyl group having substituents selected from the group consisting of an alkyl, a substituted alkyl, an aryl, a substituted aryl, or combinations thereof and at least one of the substituents is hindered, wherein the substituted silyl group is bonded to the sulfurpentafluoride group by a C—C triple bond;

ii). a sulfurpentafluoride group and a substituted silyl group having substituents selected from the group consisting of an alkyl, a substituted alkyl, an aryl, a substituted aryl, or combinations thereof and at least one of the substituents is hindered, wherein the substituted silyl group is bonded to the sulfurpentafluoride group by a C—C triple bond and wherein at least one of the substituents comprises an alkyl group;

iii) a sulfurpentafluoride group and a substituted silyl group having substituents selected from the group consisting of an alkyl, a substituted alkyl, an aryl, a substituted aryl, or combinations thereof and at least one of the substituents is hindered, wherein the substituted silyl group is bonded to the sulfurpentafluoride group by a C—C triple bond and wherein at least one of the substituents comprises t-butyl;

iv) a sulfurpentafluoride group and a substituted silyl group having substituents selected from the group consisting of an alkyl, a substituted alkyl, an aryl, a substituted aryl, or combinations thereof and at least one of the substituents is hindered, wherein the substituted silyl group is bonded to the sulfurpentafluoride group by a C—C triple bond and at least one of the substituents comprises CH₃;

v) a sulfurpentafluoride group and a substituted silyl group having substituents selected from the group consisting of an alkyl, a substituted alkyl, an aryl, a substituted aryl, or combinations thereof and at least one of the substituents is hindered, wherein the substituted silyl group is bonded to the sulfurpentafluoride group by a C—C triple bond and wherein at least one of the substitutents comprises isopropyl; and/or

vi) a sulfurpentafluoride group and a substituted silyl group having substituents selected from the group consisting of an alkyl, a substituted alkyl, an aryl, a substituted aryl, or combinations thereof and at least one of the substituents is hindered, wherein the substituted silyl group is bonded to the sulfurpentafluoride group by a C—C triple bond;

2. A compound according to embodiment 1, wherein said compound is compound (1) and R₆, R₇, and R₈ are each hydrogen, R₆ and R₈ are each hydrogen and R₇ is an unsubstituted alkyl group;

3. A compound according to embodiment 1, wherein said compound is compound (2) and:
a) R₁₁ is hydrogen, substituted or unsubstituted trialkylsilyl, an unsubstituted alkyl, or an unsubstituted aryl, or a phenyl group; or
b) R₁₁ and R₁₂ are each, independently, hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen;

4. A compound according to embodiment 1, wherein said compound is compound (3) and:
a) R₁₁ and R₁₂ are each, independently, hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen;
b) R₁₁ and R₁₂ are each hydrogen;
c) R₁₁ and R₁₂ are the same substituent group and are selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen;
d) R₁₁ and R₁₂ have the same substituent group selected from hydrogen, substituted or unsubstituted trialkylsilyl, an unsubstituted or substituted alkyl or an unsubstituted or substituted aryl;
e) R₁₁ and R₁₂ have the same substituent group selected from butyl or phenyl;
f) R₁₁ and R₁₂ are each different substituents selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen; or
g) R₁₁ and R₁₂ are each different substituents and are selected from an unsubstituted alkyl group, substituted or unsubstituted trialkylsilyl, or an unsubstituted aryl group;

5. A compound according to embodiment 1, wherein said compound is compound (4) and:
a) R₁₁ and R₁₂ have the same substituent group selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen and R₁₃ is different than R₁₁ and R₁₂ and is selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen;
b) R₁₁, R₁₂ and R₁₃ are each different and are, independently selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen;
c) R₁₁ and R₁₂ are the same and are selected from substituted or unsubstituted trialkylsilyl, an unsubstituted or substituted alkyl and R₁₃ is hydrogen;
d) R₁₁ and R₁₂ are substituted or unsubstituted trialkylsilyl, butyl or phenyl and R₁₃ is hydrogen;
e) R₁₁, R₁₂, and R₁₃ are each different substituents and are selected from an unsubstituted alkyl group, substituted or unsubstituted trialkylsilyl, an unsubstituted phenyl group or hydrogen;
f) R₁₁ is an unsubstituted alkyl group or substituted or unsubstituted trialkylsilyl, R₁₂ is an unsubstituted phenyl group, and R₁₃ is hydrogen;
g) R₁₁ is phenyl or substituted or unsubstituted trialkylsilyl, R₁₂ is alkyl, and R₁₃ is hydrogen;
h) R₁₁ and R₁₃ have the same substituent group selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen and R₁₂ is different than R₁₁ and R₁₃ and is selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen; or
i) R₁₂ and R₁₃ have the same substituent group selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen and R₁₁ is different than R₁₂ and R₁₃ and is selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen;

6. A compound according to embodiment 1, wherein said compound is compound (5) and:
a) R₁₁, R₁₄, R₁₅, R₁₆, and R₁₇ are each are, independently, hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen; or
b) R₁₁ is hydrogen, substituted or unsubstituted trialkylsilyl, an unsubstituted alkyl or an unsubstituted aryl and R₁₄, R₁₅, R₁₆, and R₁₇ are each hydrogen;

7. A compound according to embodiments 1-6, wherein a substituted substituent is substituted with a halogen, an aliphatic chain, an aryl, a ketone, an ether, a hydroxy, an alkoxy, an amino, an aldehyde, a carboxyl group, a phosphate, or a thio group;

8. A compound according to embodiment 1, wherein said compound is 3-pentafluorosulfanylfuran, 2-methyl-4-pentafluorosulfanylfuran, 3-pentafluoro-sulfanyl-4-butylfuran, 3-phenyl-4-pentafluorosulfanyl-5-butyl-isoxazole, 2,3-diphenyl-4-pentafluorosulfanyl-5-butylisooxazoline, 3,5-diphenyl-4-pentafluorosulfanylisooxazole, 4-pentafluorosulfanyl-2,3,4-tripenyl-4-isooxazoline or 2-butyl-3-pentafluorosulfanylbicyclo[2.2.1]hepta-2,5-diene;

9. A method of making a compound according to embodiments 1-8 comprising:

a) contacting an SF₅ substituted alkene with lithium hydroxide monohydrate in the presence of DMSO to form an SF₅ substituted alkyne,

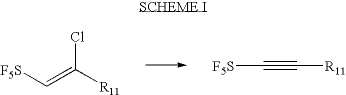

wherein R₁₁ is a substituent selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, alkoxy, ether, ketone, carboxyl, aldehyde, amino, thio, phosphate, or halogen;

b) contacting a SF₅ substituted alkynyl or alkyne with reactants and/or at least one radical initiator to form SF₅ substituted azole, azoline, bridge ring or furan compounds,

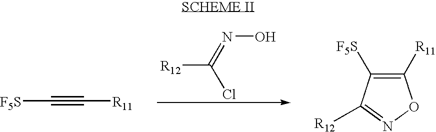

wherein R₁₁ and R₁₂ are substituents independently selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, alkoxy, ether, ketone, carboxyl, aldehyde, amino, thio, phosphate, or halogen. R₁₁ and R₁₂ are preferably and independently hydrogen, substituted or unsubstituted trialkylsilyl, unsubstituted alkyl, preferably butyl, or unsubstituted aryl, preferably phenyl and the radical initiator is selected from the group consisting of dialkylboranes, trialkylboranes, Et₃N, Et₃N-nHF, and 9-boracicyclo[3.3.1.]nonane, and mixtures thereof;

c) contacting an SF₅ substituted alkyne with an azole compound to form a SF₅ substituted furan compound,

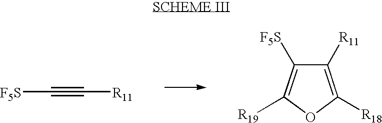

wherein R₁₁, R₁₈, and R₁₉ are each substituents independently selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, alkoxy, ether, ketone, carboxyl, aldehyde, amino, thio, phosphate, or halogen;

d) contacting a SF₅ substituted alkyne with an R₁₂, R₁₃-disubstituted aniline N-oxide to form a compound of structure (4),

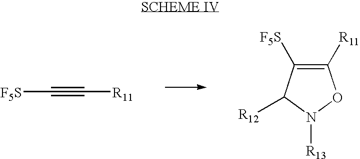

wherein R₁₁, R₁₂, and R₁₃ are substituents independently selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, alkoxy, ether, ketone, carboxyl, aldehyde, amino, thio, phosphate, or halogen;

e) contacting a SF₅ substituted alkyne with a substituted or unsubstituted cyclopentiadiene and heating the mixture to form a compound of structure (5),

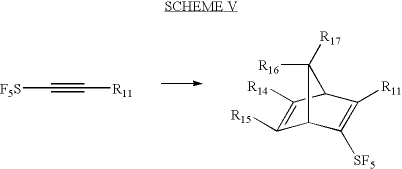

wherein R₁₁, R₁₄, R₁₅, R₁₆, and R₁₇, are each independently selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, alkoxy, ether, ketone, carboxyl, aldehyde, amino, thio, phosphate, or halogen; or

f) contacting bridged ring compounds, wherein the two rings have two atoms in common, with SF₅Cl gas and an initiator to form a SF₅ substituted furan, said initiator being selected form selected from dialkylboranes, trialkylboranes, Et₃N, Et₃N-nHF, and 9-boracicyclo[3.3.1.]nonane, or mixtures thereof;

10. A method according to embodiment 9, wherein said bridged ring compound is:

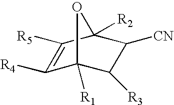

wherein R₁-R₅ are each substituents independently selected from hydrogen, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen;

11. A method according to embodiment 9, wherein said method of making a SF₅ substituted furan forms an intermediate compound of the formula:

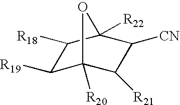

wherein R₁₈ is Cl or SF₅, R₁₉ is Cl or SF₅; and R₁₈ and R₁₉ are not the same substituent; and wherein R₂₀, R₂₁, and R₂₂ are each independently hydrogen, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen;

12. A method according to embodiment 11 wherein said first intermediate product is contacted with lithium hydroxide monohydrate in the presence of DMSO to eliminate the halogen substituent on either R₁₈ and R₁₉ to form a second intermediate product of formula:

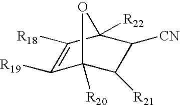

wherein R₁₈ is hydrogen or SF₅, R₁₉ is hydrogen or SF₅, R₁₈ and R₁₉ are not the same substituent and R₂₀, R₂₁, and R₂₂ each remain independently hydrogen, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, alkoxy, ether, ketone, carboxyl, aldehyde, amino, thio, phosphate, or halogen;

13. A method according to embodiment 12, further comprising heating said second intermediate compound to form compound (1);

14. A method according to embodiments 9-13, further comprising the separation or isolation of enantiomers of said compounds;

15. A method according to embodiments 9-13, further comprising the separation or isolation of intermediate compounds formed in said method;

16. A method according to embodiment 15, further comprising the separation or isolation of enantiomeric forms of said intermediate compounds; or

17. A compound of the following formula:

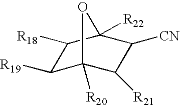

wherein R₁₈ is Cl or SF₅, R₁₉ is Cl or SF₅; and R₁₈ and R₁₉ are not the same substituent; and wherein R₂₀, R₂₁, and R₂₂ are each independently hydrogen, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen; or

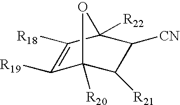

wherein R₁₈ is hydrogen or SF₅, R₁₉ is hydrogen or SF₅, R₁₈ and R₁₉ are not the same substituent and R₂₀, R₂₁, and R₂₂ each remain independently hydrogen, unsubstituted alkyl, substituted alkyl, unsubstituted alkenyl, disubstituted alkenyl, substituted alkenyl, unsubstituted alkynyl, substituted alkyl, unsubstituted aryl, substituted aryl, cycloalkanes, cycloalkenes, cyclodialkenes, alkoxy, ether, ketone, carboxyl, aldehyde, amino, thio, phosphate, or halogen.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### EXAMPLE 1

### SYNTHESIS OF 3-PENTAFLUOROSULFANYLFURAN

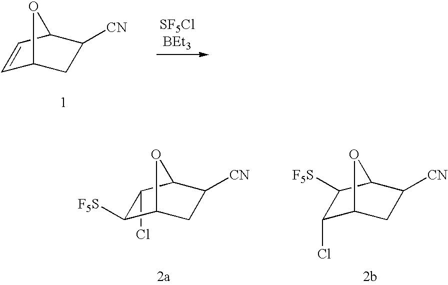

To a solution of 1 (2.2 g, 18.16 mmol) in dry CH₂Cl₂ (20 ml) was added SF₅Cl gas (8.1 g, 2.7 equiv.) at −40° C. 0.26 ml (0.1 equiv.) of BEt₃ (triethylborane) was added dropwise to the mixture over 30 minutes at −40° C. After stirring for 2 hours at a temperature between −40° C. to −30° C., extra SF₅Cl gas (2.0 g, 0.67 equiv.) was added to the reaction mixture at −40° C., and then 0.26 ml of BEt₃ was added to the mixture dropwise over 30 minutes at −40° C. The reaction mixture was stirred for 2 hours at a temperature between −40° C. to −30° C., and then left in a freezer (−25° C.) overnight. The resultant mixture was poured into ice water, neutralized with NaHCO₃, and extracted twice with CH₂Cl₂. The organic layers were combined, washed with brine, and dried over MgSO₄. The solvent was removed under reduced pressure.

The crude product was purified by silica gel column chromatography (elution with n-Hexane/Ethyl acetate=4/1). 4.4 g of a mixture of 2a and 2b (2a:2b=2:1) was obtained (86%), and the mixture had the following characteristics:

2 (2a:2b=2:1); ¹HNMR, δ 2.10-2.38 (m, 2a; 2H, —CH₂— and 2b; 1H, —CH₂—), 2.77 (dd, 2b; 1H, —CH₂—, J=13.5, 9.0 Hz), 2.91 (dd, 2b; 1H, —CH(CN)—, J=9.0, 3.9 Hz), 3.53 (dd, 2a; 1H, —CH(CN)—, J=8.7, 5.1 Hz), 3.74-3.90 (m, 2a; 1H, —CH(SF₅)— and 2b; 1H, —CH(SF₅)—) 4.72 (t, 2b; 1H, —CHCl—, J=5.1 Hz), 4.76 (dd, 2a; 1H, —CHCl—, J=5.1 Hz), 4.87 (t, 2b; 1H, bridge-CH—, J=5.3 Hz), 4.96 (d, 2a; 1H, bridge-CH—, J=5.3 Hz), 5.27 (d, 2a; 1H, bridge-CH—, J=5.3 Hz), 5.32 (s, 2b; 1H, bridge-CH—); ¹⁹F NMR, δ 58.9 (d, 2a; 4F, J=152 Hz), 59.6 (d, 2b; 4F, J=153 Hz), 81.3 (p, 2b; 1F, J=153 Hz), 81.5 (p, 2a; 1F, J=152 Hz)

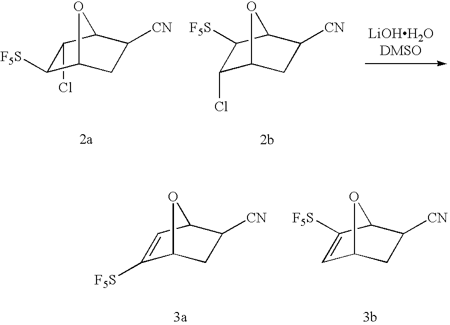

To a solution of 2 (2a:2b=2:1, 1.0 g, 3.52 mmol) in DMSO (60 ml) was added LiOH.H₂O (0.74 g, 17.6 mmol) at room temperature. The mixture was stirred for 1 hour at room temperature. The resultant mixture was poured into ice-water, neutralized with NaHCO₃, and extracted twice with ethyl acetate. The organic layers were combined, washed with water and brine, and then dried over MgSO₄. The solvent was removed under reduced pressure. The crude product was purified by silica gel column chromatography (elution with n-Hexane/Ethyl acetate=4/1). 0.54 g of 3a and 0.30 g of 3b were obtained (96%), and the mixture of 3a and 3b had the following characteristics:

3a; ¹H NMR, δ 2.11 (dd, 1H, —CH₂—, J=12.0, 8.4 Hz), 2.29 (dt, 1H, —CH₂—, J=12.0, 4.0 Hz), 2.67 (dd, 1H, —CH(CN)—, J=8.4, 4.0 Hz), 5.36 (s, 1H, bridge-CH—), 5.37 (d, 1H, bridge-CH—, J=4.0), 6.74 (s, 1H, —CH═C(SF₅)—); ¹⁹F NMR, δ 65.9 (d, 4F, J=161 Hz), 80.2 (p, 1F, J=161 Hz); ¹³C NMR, δ 27.6, 31.5, 79.1, 81.9, 120.5, 135.0, 161.2

3b; ¹H NMR, δ 1.99 (dd, 1H, —CH₂—, J=12.0, 8.7 Hz), 2.26-2.40 (m, 1H, —CH₂—), 2.78 (dd, 1H, —CH(CN)—, J=8.7, 3.9 Hz), 5.32 (bs, 1H, bridge-CH—), 5.44 (s, 1H, bridge-CH—, J=4.0), 6.87 (s, 1H, —CH═C(SF₅)—); ¹⁹F NMR, δ 66.2 (d, 4F, J=161 Hz), 80.2 (p, 1F, J=161 Hz); ¹³C NMR, δ 28.2, 31.1, 79.0, 81.8, 120.5, 139.0, 157.8

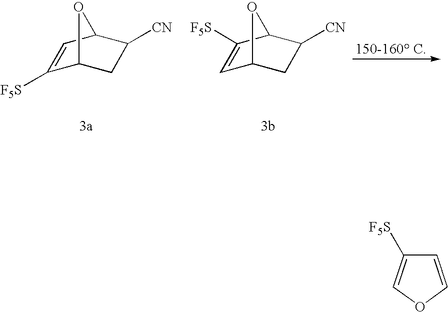

2.86 g of a mixture of 3 (3a:3b=2:1) was heated in a sealed tube at a temperature between 150° C. to 160° C. for 30 minutes. The reaction mixture was cooled to room temperature. The reaction mixture contained 1.6 g (71%) of 3-pentafluorosulfanylfuran, as determined by NMR. The reaction mixture was purified by silica gel column chromatography (elution with n-pentane). 1.40 g of 3-pentafluorosulfanylfuran was obtained (57%), and it had the following characteristics:

3-pentafluorosulfanylfuran; ¹H NMR, δ 6.67 (m, 1H), 7.42 (s, 1H), 7.84 (s, 1H); ¹⁹F NMR, δ 70.4 (d, 4F, J=165 Hz), 82.4 (p, 1F, J=165 Hz)

### EXAMPLE 2

### SYNTHESIS OF 2-METHYL-4-PENTAFLUOROSULFANYLFURAN

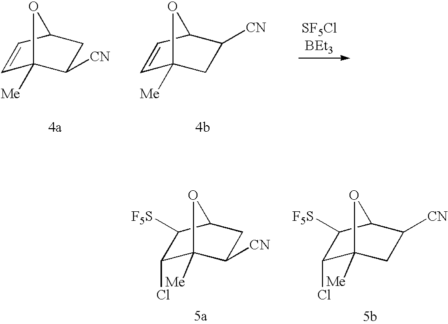

To a solution of a mixture of 4 (4a:4b=4:1, 1.0 g, 7.4 mmol) in dry CH₂Cl₂ (10 ml) was added SF₅Cl gas (5.4 g, 4.5 equiv.) at a temperature between −40° C. to −50° C. 0.1 ml (0.1 equiv.) of BEt₃ was added dropwise to the mixture over 30 minutes at −40° C., stirred for 4 hours at −40° C. to −30° C., and then left in a freezer at −25° C. overnight. The reaction mixture was poured into ice water, neutralized with NaHCO₃, and extracted twice with CH₂Cl₂. The organic layers were combined, washed with brine, and dried over MgSO₄. The solvent was removed under reduced pressure.

The crude product was purified by silica gel column chromatography (elution with n-Hexane/Ethyl acetate=4/1). 2.0 g of a mixture of 5a and 5b (5a:5b=4:1) was obtained (90%), and the mixture had the following characteristics:

5(5a:5b=4:1); ¹H NMR, δ 1.64 (s, 5b; 3H, Me), 1.77 (s, 5a; 3H, Me), 1.80-1.90 (m, 5b; 1H, —CH₂—), 2.22-2.24 (m, 5a; 2H, —CH₂—), 2.80 (dd, 5b; 1H, —CH₂—, J=13.2, 9.0 Hz), 2.95 (dd, b; 1H, —CH(CN)—, J=9.0, 4.0 Hz), 3.49 (dd, 5a; 1H, —CH(CN)—, J=8.9, 6.2 Hz), 3.82-4.00 (m, 5a; 1H, —CH(SF₅)— and 5b; 1H, —CH(SF₅)—) 4.36 (m, 5b; 1H, —CHCl—), 4.45 (d, 5a; 1H, —CHCl—, J=5.4 Hz), 5.14 (d, 5a; 1H, bridge-CH—, J=5.7 Hz), 5.22 (s, 5b; 1H, bridge-CH—); ¹⁹F NMR, δ 58.6 (d, 5a; 4F, J=156 Hz), 59.3 (d, 5b; 4F, J=156 Hz), 81.4 (p, 5b; 1F, J=156 Hz), 81.8 (p, 5a; 1F, J=156 Hz)

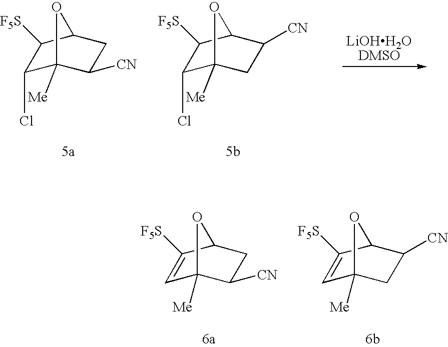

To a solution of 5 (5a:5b=4:1, 2.0 g, 6.7 mmol) in DMSO (7.5 ml) was added LiOH.H₂O (1.55 g, 36.9 mmol) at room temperature. The mixture was stirred for 1 hour at room temperature. The resultant mixture was poured into ice-water, neutralized with NaHCO₃, and extracted twice with Ethyl acetate. The organic layers were combined, washed with water and brine, and then dried over MgSO₄. The solvent was removed under reduced pressure. The crude product was purified by silica gel column chromatography (elution with n-Hexane/Ethyl acetate=4/1). 1.66 g of a mixture of 6 (6a, 6b, and one more isomer) was obtained (95%), and the mixture had the following characteristics:

6; ¹H NMR, δ 1.76, 1.82, 1.88 (s, 3H, —CH₃), 1.98-2.88 (m, 2H, —CH₂—), 5.23-5.36 (m, 1H, bridge-CH—), 6.54, 6.67, 6.72 (s, 1H, —CH═C(SF₅)—); ¹⁹F NMR, δ 65.3, 65.6, 65.7 (d, 4F, J=161 Hz), 78.0-81.2 (p, 1F)

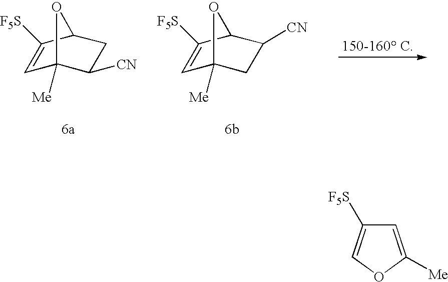

1.12 g of a mixture of 6 (6a, 6b and one more isomer) was heated in sealed tube at a temperature between 150° C. to 160° C. for 30 minutes. The reaction mixture was cooled to room temperature. The reaction mixture contained 0.78 g (87%) of 3-pentafluorosulfanylfuran, as determined by NMR. The reaction mixture was purified silica gel column chromatography (elution with n-pentane). 0.70 g of 2-methyl-4-pentafluorosulfanylfuran was obtained (78%), and it had the following characteristics:

2-methyl-4-pentafluorosulfanylfuran; ¹H NMR, δ 2.29 (s, 3H), 6.25 (m, 1H), 7.65 (s, 1H); ¹⁹F NMR, δ 69.9 (d, 4F, J=163 Hz), 83.1 (p, 1F, J=163 Hz)

### EXAMPLE 3

### SYNTHESIS OF 1-PENTAFLUOROSULFANYLHEXYNE

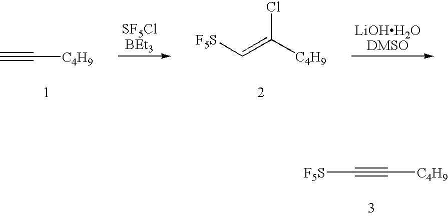

To a solution of 1 (2.2 g, 18.16 mmol) in dry n-Hexane (30 ml) was added SF₅Cl gas (8.1 g, 2.7 equiv.) at −40° C. 0.26 ml (0.1 equiv.) of BEt₃ was added dropwise to the mixture over 30 minutes at −40° C. The reaction mixture was stirred for 1 hour at −40° C. to −30° C. and then was warmed to room temperature. The resultant mixture was poured into water, neutralized with NaHCO₃, and extracted twice with n-Hexane. The organic layers were combined, washed with brine, and dried over MgSO₄. The solvent was removed under reduced pressure. 4.4 g of 2 was obtained (86%), and 2 had the following characteristics:

2; ¹H NMR, δ 0.95 (t, 3H, —CH₃, J=7.2 Hz), 1.25-1.45 (m, 2H, —CH₂—), 1.45-1.70 (m, 2H, —CH₂—), 2.68 (t, 2H, ═CCl—CH₂—, J=7.7 Hz), 6.60 (p, 1H, ═CH(SF₅), J=8.4 Hz); ¹⁹F NMR, δ 66.9 (d, 4F, J=157 Hz), 82.5 (p, 1F, J=157 Hz)

To a solution of 2 (1.0 g, 3.52 mmol) in DMSO (60 ml) was added LiOH.H₂O (0.74 g, 17.6 mmol) at room temperature. The mixture was stirred for 2 hours at room temperature. The resultant mixture was poured into ice-water, neutralized with 2M HCl, and extracted twice with ethyl ether. The organic layers were combined, washed with water and brine, and then dried over MgSO₄. The solvent was removed by distillation, and 3 was distilled at 120-125° C. 5.8 g of 3 was obtained (68%), and it had the following characteristics:

3; ¹H NMR, δ 0.94 (t, 3H, —CH₃, J=7.2 Hz), 1.43 (m, 2H, —CH₂—), 1.51-1.64 (m, 2H, —CH₂—), 2.32 (m, 2H, SF₅CCH₂—); ¹⁹F NMR, δ 77.8 (p, 1F, J=169 Hz), 83.0 (p, 4F, J=169 Hz)

### EXAMPLE 4

### SYNTHESIS OF 3-PHENYL-4-PENTAFLUOROSULFANYL-5-BUTYLISOOXAZOLE

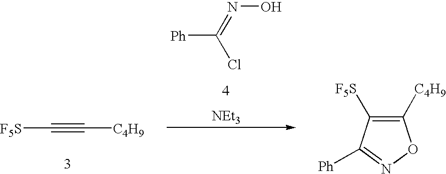

To a solution of 3 (0.21 g, 1.0 mmol) in THF (5 ml) was added 0.77 g (5.0 mmol, 5 equiv.) of Benzohydroxyiminoyl chloride (4). A solution of NEt₃ (0.51 g, 5.0 mmol, 5 equiv.) in 5 ml of THF was added dropwise to the mixture at room temperature over 30 minutes. After stirring for 16 hours at room temperature, five more equivalents of 4 and NEt₃ were added dropwise. After stirring for 20 hours at room temperature, ten more equivalents of 4 and NEt₃ were added dropwise. The reaction mixture was stirred for 3 more days at room temperature. The resultant mixture was poured into water and extracted twice with ethyl acetate. The organic layers were combined, washed with brine, and then dried over MgSO₄. The solvent was removed under reduced pressure. The crude product was purified by silica gel column chromatography (elution with n-Hexane/CHCl₃=7/3). 0.15 g of 3-phenyl-4-pentafluorosulfanyl-5-butylisooxazole was obtained (45%), and it had the following characteristics:

3-phenyl-4-pentafluorosulfanyl-5-butylisooxazole; ¹H NMR, δ 0.99 (t, 3H, —CH₃, J=7.2 Hz), 1.47 (m, 2H, —CH₂—), 1.81 (p, 2H, —CH₂—, J=7.8 Hz), 3.04 (t, 2H, —CH₂—, J=7.8 Hz), 7.3-7.6 (m, 5H, Ph); ¹⁹F NMR, δ 75.1 (d, 4F, J=165 Hz), 83.1 (p, 1F, J=165 Hz)

### EXAMPLE 5

### SYNTHESIS OF 2,3-DIPHENYL-4-PENTAFLUOROSULFANYL-5-BUTYLISOOXAZOLINE

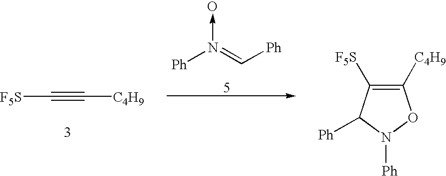

To a solution of 3 (0.21 g, 1.0 mmol) in THF (5 ml) was added 0.50 g (2.0 mmol, 2 equiv.) of N-benzylideneaniline N-oxide (5). The reaction mixture was stirred for 16 hours at room temperature. The solvent was removed under reduced pressure. The residue was purified by silica gel column chromatography (elution with n-hexane/CHCl₃=7/3). 0.27 g of 2,3-diphenyl-4-pentafluorosulfanyl-5-butyl-4-isooxazoline was obtained (67%), and it had the following characteristics:

2,3-diphenyl-4-pentafluorosulfanyl-5-butylisooxazoline; ¹H NMR, δ 1.00 (t, 3H, —CH₃, J=7.4 Hz), 1.40-1.60 (m, 2H, —CH₂—), 1.70-1.84 (m, 2H, —CH₂—), 2.75 (t, 2H, —CH₂—, J=7.8 Hz), 5.60 (s, 1H), 7.28-7.48 (m, 5H, Ph); ¹⁹F NMR, δ 73.5 (d, 4F, J=161 Hz), 86.7 (p, 1F, J=161 Hz)

### EXAMPLE 6

### SYNTHESIS OF 1-PENTAFLUOROSULFANYL-2-PHENYLACETYLENE

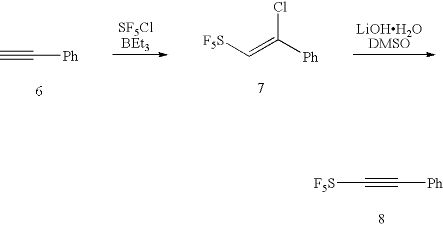

To a solution of 6 (2.2 ml, 20.0 mmol) in dry n-hexane (30 ml) was added SF₅Cl gas (4.0 g, 1.2 equiv.) at −40° C. 0.29 ml (0.1 equiv.) of BEt₃ was added dropwise to the mixture over 30 minutes at −40° C. The reaction mixture was stirred for 1 hour at −40° C. to −30° C. and then was warmed to room temperature. The resultant mixture was poured into water, neutralized with NaHCO₃, and extracted twice with n-hexane. The organic layers were combined, washed with brine, and dried over MgSO₄. The solvent was removed under reduced pressure. The residue was purified by silica gel column chromatography (elution with n-hexane). 1.5 g of 7 was obtained (28%), and 7 had the following characteristics:

7; ¹H NMR, δ 6.93 (p, 1H, ═CH(SF₅), J=7.7 Hz), 7.30-7.44 (m, 5H, Ph); ¹⁹F NMR, δ 68.5 (d, 4F, J=161 Hz), 80.9 (p, 1F, J=161 Hz)

To a solution of 7 (1.5 g, 5.67 mmol) in DMSO (5 ml) was added LiOH.H₂O (1.2 g, 28.6 mmol, 5 equiv.) at room temperature. The mixture was stirred for 2 hours at room temperature. The resultant mixture was poured into ice-water, neutralized with 2M HCl, and extracted twice with ethyl ether. The organic layers were combined, washed with brine, and then dried over MgSO₄. The solvent was removed under reduced pressure. The residue was purified by silica gel column chromatography (elution with n-hexane). 1.2 g of 8 was obtained (93%), and 8 had the following characteristics:

8; ¹H NMR, δ 7.30-7.50 (m, 3H), 7.55 (d, 2H, J=8.3 Hz); ¹⁹F NMR, δ 76.7 (p, 1F, J=170 Hz), 83.1 (p, 4F, J=170 Hz)

### EXAMPLE 7

### SYNTHESIS OF 3,5-DIPHENYL-4-PENTAFLUOROSULFANYLISOOXAZOLE

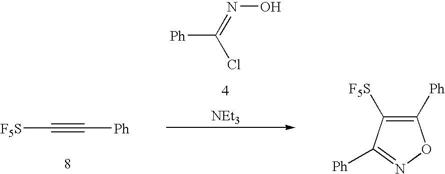

To a solution of 8 (0.50 g, 2.2 mmol) in THF (5 ml) was added 0.68 g (4.4 mmol, 2 equiv.) of benzohydroxyiminoyl chloride (4). A solution of NEt₃ (0.44 g, 4.4 mmol, 2 equiv.) in 5 ml of THF was added dropwise to the mixture at room temperature over 30 minutes. After stirring for 16 hours at room temperature, the 3.5 equivalent of 4 and NEt₃ were added dropwise. The reaction mixture was stirred for 20 hours at room temperature. The resultant mixture was poured into water and extracted twice with ethyl acetate. The organic layers were combined, washed with brine, and then dried over MgSO₄. The solvent was removed under reduced pressure. The crude product was purified by silica gel column chromatography (elution with n-hexane/CHCl₃=7/3). 0.4 g of 3,5-diphenyl-4-pentafluorosulfanylisooxazole was obtained (53%), and it had the following characteristics:

3,5-diphenyl-4-pentafluorosulfanylisooxazole; ¹H NMR, δ 0.99 (t, 3H, —CH₃, J=7.2 Hz), 1.47 (m, 2H, —CH₂—), 1.81 (p, 2H, —CH₂—, J=7.8 Hz), 3.04 (t, 2H, —CH₂—, J=7.8 Hz), 7.3-7.6 (m, 5H, Ph); ¹⁹F NMR, δ 75.1 (d, 4F, J=165 Hz), 83.1 (p, 1F, J=165 Hz)

### EXAMPLE 8

### SYNTHESIS OF 4-PENTAFLUOROSULFANYL-2,3,5-TRIPHENYL-4-ISOOXAZOLINE

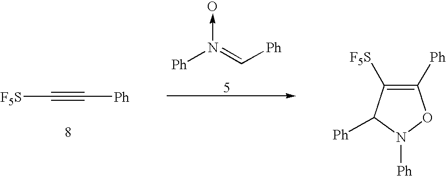

To a solution of 8 (0.36 g, 1.57 mmol) in THF (5 ml) was added 0.62 g (3.14 mmol, 2 equiv.) of N-benzylideneaniline N-oxide (5). The reaction mixture was stirred for 20 hours at room temperature. The solvent was removed under reduced pressure. The residue was purified by silica gel column chromatography (elution with n-hexane/CHCl₃=7/3). 0.6 g of 4-pentafluorosulfanyl-2,3,5-triphenyl-4-isooxazoline was obtained (90%), and it had the following characteristics:

4-pentafluorosulfanyl-2,3,5-triphenyl-4-isooxazoline; ¹H NMR, 5.80 (s, 1H), 7.16 (t, 1H), 7.24 (d, 2H), 7.35-7.60 (m, 10H), 7.75 (dd, 2H, J=7.5, 2.0 Hz); ¹⁹F NMR, δ 74.8 (d, 4F, J=161 Hz), 85.1 (p, 1F, J=161 Hz)

### EXAMPLE 9

### SYNTHESIS OF 2-BUTYL-3-PENTAFLUOROSULFANYL-2,5-NORBORNADIENE

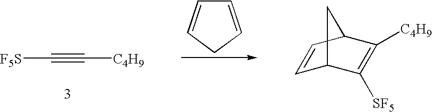

A mixture of 3 (0.5 g, 2.4 mmol) and freshly distilled cyclopentidiene (1.58 g, 24.0 mmol, 10 equiv.) was heated in a sealed tube at 120° C. for 18 hours. The reaction mixture was cooled to room temperature. The reaction mixture contained 0.46 g (70%) of 2-butyl-3-pentafluorosulfanyl-2,5-norbornadiene, as determined by NMR. Unreacted cyclopentadiene and dicyclopentadiene, which were formed during the reaction, was removed under reduced pressure. The residue was purified by silica gel column chromatography (elution with n-hexane). 0.3 g of 2-butyl-3-pentafluorosulfanyl-2,5-norbornadiene was obtained (50%), and it had the following characteristics:

2-butyl-3-pentafluorosulfanyl-2,5-norbornadiene; ¹H NMR, 0.91 (t, 3H, J=7.2 Hz), 1.20-1.60 (m, 4H), 1.97 (d, 1H, J=6.6 Hz), 2.18 (dt, 1H, J=6.6, 1.7 Hz), 2.43 (m, 2H), 3.51 (bs, 1H), 3.88 (bs, 1H), 6.78 (m, 1H), 6.90 (m, 1H); ¹⁹F NMR, δ 64.9 (d, 4F, J=161 Hz), 87.5 (p, 1F, J=161 Hz)

### EXAMPLE 10

### SYNTHESIS OF 3-BUTYL-4-PENTAFLUOROSULFANYLFURAN

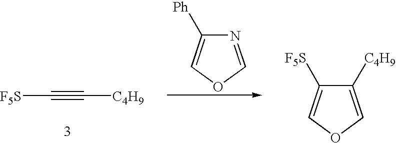

A mixture of 3 (0.36 g, 1.7 mmol) and 4-phenyloxazole (0.5 g, 3.4 mmol, 2.0 equiv.) was heated in sealed tube at 180° C. to 190° C. for 20 hours. The reaction mixture was cooled to room temperature. The reaction mixture contained 0.30 g (69%) of 3-butyl-4-pentafluorosulfanylfuran, as determined by NMR, and purified by silica gel column chromatography (elution with n-Hexane). 0.25 g of 3-bButyl-4-pentafluorosulfanylfuran was obtained (58%), and it had the following characteristics:

3-butyl-4-pentafluorosulfanylfuran; ¹H NMR, 0.95 (t, 3H, J=7.3 Hz), 1.46 (m, 2H), 1.50-1.64 (m, 2H), 2.53 (t, 2H, J=8.0), 7.16 (d, 1H, J=0.9 Hz), 7.79 (s, 1H); ¹⁹F NMR, δ 73.4 (d, 4F, J=161 Hz), 84.2 (p, 1F, J=161 Hz)

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

1-17. (canceled)

18. A compound selected from:
compound (1): 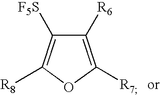
compound (3): 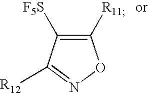
compound (4): 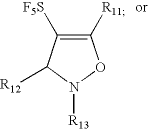
compound (5) 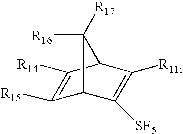
wherein R₆, R₇, R₈, R₁₁, R₁₂, R₁₃, R₁₄, R₁₅, R₁₆, and R₁₇ are, independently, hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkynyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen;

19. The compound according to claim 18, wherein said substituted trialkylsilyl, substituted alkyl, disubstituted alkenyl, substituted alkenyl, substituted alkynyl, and substituted aryl are substituted with halogen, aliphatic chain, aryl, ketone, ether, hydroxy, alkoxy, amino, aldehyde, carboxyl group, phosphate, or thio group.

20. The compound according to claim 19, wherein said compound is compound (1) and R₆, R₇, and R₈ are each hydrogen, or R₆ and R₈ are each hydrogen and R₇ is an unsubstituted alkyl group.

21. The compound according to claim 19, wherein said compound is compound (3) and:
a) R₁₁ and R₁₂ are each, independently, hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkynyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen;
b) R₁₁ and R₁₂ are each hydrogen;
c) R₁₁ and R₁₂ are the same substituent group and are selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkynyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen;
d) R₁₁ and R₁₂ have the same substituent group selected from hydrogen, substituted or unsubstituted trialkylsilyl, an unsubstituted or substituted alkyl or an unsubstituted or substituted aryl;
e) R₁₁ and R₁₂ have the same substituent group selected from butyl or phenyl;
f) R₁₁ and R₁₂ are each different substituents selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkynyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen; or
g) R₁₁ and R₁₂ are each different substituents and are selected from an unsubstituted alkyl group, substituted or unsubstituted trialkylsilyl, or an unsubstituted aryl group.

22. The compound according to claim 19, wherein said compound is compound (4) and:
a) R₁₁ and R₁₂ have the same substituent group selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkynyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen and R₁₃ is different than R₁₁ and R₁₂ and is selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkynyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen;
b) R₁₁, R₁₂ and R₁₃ are each different and are, independently selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkynyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen;
c) R₁₁ and R₁₂ are the same and are selected from substituted or unsubstituted trialkylsilyl, an unsubstituted or substituted alkyl and R₁₃ is hydrogen;
d) R₁₁ and R₁₂ are substituted or unsubstituted trialkylsilyl, butyl or phenyl and R₁₃ is hydrogen;
e) R₁₁, R₁₂, and R₁₃ are each different substituents and are selected from an unsubstituted alkyl group, substituted or unsubstituted trialkylsilyl, an unsubstituted phenyl group or hydrogen;
f) R₁₁ is an unsubstituted alkyl group or substituted or unsubstituted trialkylsilyl, R₁₂ is an unsubstituted phenyl group, and R₁₃ is hydrogen;
g) R₁₁ is phenyl or substituted or unsubstituted trialkylsilyl, R₁₂ is alkyl, and R₁₃ is hydrogen;
h) R₁₁ and R₁₃ have the same substituent group selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkynyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen and R₁₂ is different than R₁₁ and R₁₃ and is selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkynyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen; or
i) R₁₂ and R₁₃ have the same substituent group selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkynyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen and R₁₁ is different than R₁₂ and R₁₃ and is selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkynyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen.

23. The compound according to claim 19, wherein said compound is compound (5) and:
a) R₁₁, R₁₄, R₁₅, R₁₆, and R₁₇ are each are, independently, hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkynyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen; or
b) R₁₁ is hydrogen, substituted or unsubstituted trialkylsilyl, an unsubstituted alkyl or an unsubstituted aryl and R₁₄, R₁₅, R₁₆, and R₁₇ are each hydrogen.

24. The compound according to claim 18, wherein a substituted substituent is substituted with a halogen, an aliphatic chain, an aryl, a ketone, an ether, a hydroxy, an alkoxy, an amino, an aldehyde, a carboxyl group, a phosphate, or a thio group.

25. The compound of claim 18, wherein said compound is 3-pentafluorosulfanylfuran, 2-methyl-4-pentafluorosulfanylfuran, 3-pentafluoro-sulfanyl-4-butylfuran, 3-phenyl-4-pentafluorosulfanyl-5-butyl-isoxazole, 2,3-diphenyl-4-pentafluorosulfanyl-5-butylisooxazoline, 3,5-diphenyl-4-pentafluorosulfanylisooxazole, 4-pentafluorosulfanyl-2,3,4-tripenyl-4-isooxazoline or 2-butyl-3-pentafluorosulfanylbicyclo[2.2.1]hepta-2,5-diene.

26. A method of making a compound according to claim 18 comprising:
a) contacting an SF₅ substituted alkene with lithium hydroxide monohydrate in the presence of DMSO to form an SF₅ substituted alkyne, 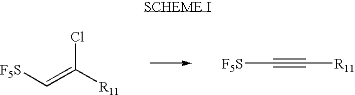
wherein R₁₁ is a substituent selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkynyl, unsubstituted aryl, substituted aryl, cycloalkanes, cycloalkenes, cyclodialkenes, alkoxy, ether, ketone, carboxyl, aldehyde, amino, thio, phosphate, or halogen;
b) contacting a SF₅ substituted alkynyl or alkyne with reactants and/or at least one radical initiator to form SF₅ substituted azole, azoline, bridge ring or furan compounds, 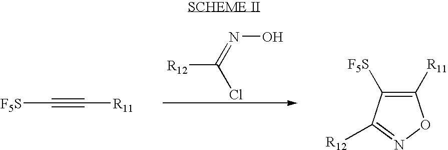
wherein R₁₁ and R₁₂ are substituents independently selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkynyl, unsubstituted aryl, substituted aryl, cycloalkanes, cycloalkenes, cyclodialkenes, alkoxy, ether, ketone, carboxyl, aldehyde, amino, thio, phosphate, or halogen. R₁₁ and R₁₂ are preferably and independently hydrogen, substituted or unsubstituted trialkylsilyl, unsubstituted alkyl, preferably butyl, or unsubstituted aryl, preferably phenyl and the radical initiator is selected from the group consisting of dialkylboranes, trialkylboranes, Et₃N, Et₃N-nHF, and 9-boracicyclo[3.3.1.]nonane, and mixtures thereof;
c) contacting an SF₅ substituted alkyne with an azole compound to form a SF₅ substituted furan compound, 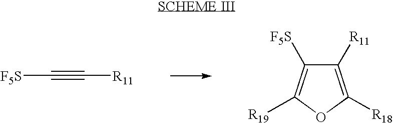
wherein R₁₁, R₁₈, and R₁₉ are each substituents independently selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkynyl, unsubstituted aryl, substituted aryl, cycloalkanes, cycloalkenes, cyclodialkenes, alkoxy, ether, ketone, carboxyl, aldehyde, amino, thio, phosphate, or halogen;
d) contacting a SF₅ substituted alkyne with an R₁₂, R₁₃-disubstituted aniline N-oxide to form a compound of structure (4), 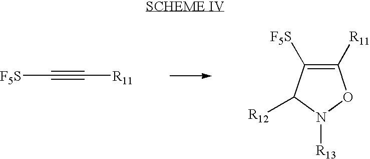
wherein R₁₁, R₁₂, and R₁₃ are substituents independently selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkynyl, unsubstituted aryl, substituted aryl, cycloalkanes, cycloalkenes, cyclodialkenes, alkoxy, ether, ketone, carboxyl, aldehyde, amino, thio, phosphate, or halogen;
e) contacting a SF₅ substituted alkyne with a substituted or unsubstituted cyclopentiadiene and heating the mixture to form a compound of structure (5), 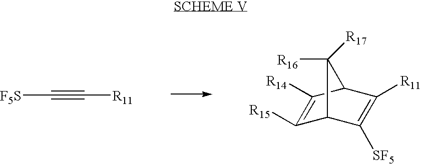
wherein R₁₁, R₁₄, R₁₅, R₁₆, and R₁₇, are each independently selected from hydrogen, substituted or unsubstituted trialkylsilyl, unsubstituted alkyl, substituted alkyl, unsubstituted alkenyl, disubstituted alkenyl, substituted alkenyl, unsubstituted alkynyl, substituted alkynyl, unsubstituted aryl, substituted aryl, cycloalkanes, cycloalkenes, cyclodialkenes, alkoxy, ether, ketone, carboxyl, aldehyde, amino, thio, phosphate, or halogen; or
f) contacting bridged ring compounds, wherein the two rings have two atoms in common, with SF₅Cl gas and an initiator to form a SF₅ substituted furan, said initiator being selected form selected from dialkylboranes, trialkylboranes, Et₃N, Et₃N-nHF, and 9-boracicyclo[3.3.1.]nonane, or mixtures thereof.

27. The method according to claim 26, wherein said bridged ring compound is: 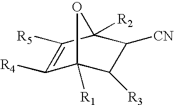
wherein R₁-R₅ are each substituents independently selected from hydrogen, unsubstituted alkyl, substituted alkyl, unsubstituted alkenyl, disubstituted alkenyl, substituted alkenyl, unsubstituted alkynyl, substituted alkynyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen.

28. The method according to claim 27, further comprising the separation or isolation of enantiomers of said compounds.

29. The method according to claim 27, further comprising the separation or isolation of intermediate compounds formed in said method.

30. The method according to claim 29, further comprising the separation or isolation of enantiomeric forms of said intermediate compounds.

31. The method according to claim 26, wherein said method of making a SF₅ substituted furan forms an intermediate compound of the formula: 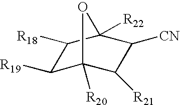
wherein R₁₈ is Cl or SF₅, R₁₉ is Cl or SF₅; and R₁₈ and R₁₉ are not the same substituent; and wherein R₂₀, R₂₁, and R₂₂ are each independently hydrogen, unsubstituted alkyl, substituted alkyl, unsubstituted alkenyl, disubstituted alkenyl, substituted alkenyl, unsubstituted alkynyl, substituted alkynyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen.

32. The method according to claim 31, wherein said first intermediate product is contacted with lithium hydroxide monohydrate in the presence of DMSO to eliminate the halogen substituent on either R₁₈ and R₁₉ to form a second intermediate product of formula: 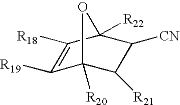
wherein R₁₈ is hydrogen or SF₅, R₁₉ is hydrogen or SF₅, R₁₈ and R₁₉ are not the same substituent and R₂₀, R₂₁, and R₂₂ each remain independently hydrogen, unsubstituted alkyl, substituted alkyl, unsubstituted alkenyl, disubstituted alkenyl, substituted alkenyl, unsubstituted alkynyl, substituted alkynyl, unsubstituted aryl, substituted aryl, cycloalkanes, cycloalkenes, cyclodialkenes, alkoxy, ether, ketone, carboxyl, aldehyde, amino, thio, phosphate, or halogen.

33. The method according to claim 32, further comprising heating said second intermediate compound to form compound (1).

34. The method according to claim 33, further comprising the separation or isolation of enantiomers of said compounds.

35. The method according to claim 33, further comprising the separation or isolation of intermediate compounds formed in said method.

36. The method according to claim 35, further comprising the separation or isolation of enantiomeric forms of said intermediate compounds.

37. A compound of the following formula: 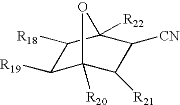
wherein R₁₈ is Cl or SF₅, R₁₉ is Cl or SF₅; and R₁₈ and R₁₉ are not the same substituent; and wherein R₂₀, R₂₁, and R₂₂ are each independently hydrogen, unsubstituted alkyl, substituted alkyl, unsubstituted alkenyl, disubstituted alkenyl, substituted alkenyl, unsubstituted alkynyl, substituted alkynyl, unsubstituted aryl, substituted aryl, cycloalkanes, cycloalkenes, cyclodialkenes, hydroxy, alkoxy, ether, ketone, carboxyl, aldehyde, amino, thio, phosphate, or halogen; or 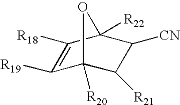
wherein R₁₈ is hydrogen or SF₅, R₁₉ is hydrogen or SF₅, R₁₈ and R₁₉ are not the same substituent and R₂₀, R₂₁, and R₂₂ each remain independently hydrogen, unsubstituted alkyl, substituted alkyl, unsubstituted alkenyl, disubstituted alkenyl, substituted alkenyl, unsubstituted alkynyl, substituted alkynyl, unsubstituted aryl, substituted aryl, cycloalkanes, cycloalkenes, cyclodialkenes, alkoxy, ether, ketone, carboxyl, aldehyde, amino, thio, phosphate, or halogen.
